(19) United States
(12) Patent Application Publication
(10) Pub. No.: US 2007/0141727 A1
(43) Pub. Date: Jun. 21, 2007

(54) Luminescent metallic cluster particles and uses thereof

(75) Inventors: Lei Huang, Duluth, GA (US); Kaiyuan Yang, Cumming, GA (US); Xuedong Song, Roswell, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/304,864

(22) Filed: Dec. 15, 2005

(51) Int. Cl. G01N 33/553

(52) U.S. Cl. 436/526

(57) ABSTRACT

A luminescent particle having a luminescent core structure formed from at least one type of luminescent metallic cluster complex and a shell that covers said core structure is described. The luminescent core structure includes one or more aggregates of metal cluster complexes. The shell has one or more layers of thin films formed predominately of organic or inorganic polymers, hydrid polymers, or combinations thereof. The luminescent particles can be adapted for optical detection or imaging applications. Methods of fabricating the various iterations of the luminescent particles are also provided.

### FIELD OF INVENTION

The present invention relates to luminescent metallic cluster particles that may be used in optical detection or imaging applications. In particular, the invention pertains to particles that contain a core that includes at least one kind of luminescent metallic cluster complex and a shell of an organic or inorganic material, which at least partially covers or encapsulates the core structure.

### BACKGROUND

Luminescent metallic cluster complexes have attracted much attention in recent years, in part due to their potential use in a wide variety of applications, such as in optical light-emitting diode (OLED) display technology as dopant emitters, in solar photoconversion chemistry as chromophores, and in sensor development for luminescence detection. Attractive in this regard are polynuclear metal-metal complexes that possess intense, long-lived luminescence in the solid state at ambient temperatures with emission energies spanning the visible spectrum. Different excited-state assignments have been made for these systems, including metal-centered or cluster-based (MC), ligand-to-metal charge transfer (LMCT), and intraligand (IL), with aggregation through metalophilic interactions thought to play an important role in producing the emissive state.

Many luminescent metallic cluster complexes reported in the scientific literature, have several drawbacks for many applications. For instance, the complexes are dispensable in water or not compatible with biological system. This aqueous incompatibility prevents the complexes from being used directly for biological detection and imaging. Moreover, since it is believed that the nature of the luminescence is phosphorescence, the complexes may also be susceptible to easy quenching from exposure to oxygen, nitrogen oxide, or other oxidative or reactive molecules.

Photoluminescent molecules also tend to be rather delicate and can be affected by polar environments, such as in aqueous or other liquid mediums. In some cases, photoluminescent molecules, particularly metallic cluster complexes, may not be dispersible in aqueous or desired liquid media, which impose additional constraints to their use in certain applications. To extend the use of luminescent metallic cluster complexes into wider fields of use, such as the realm of biological or chemical assays, the metallic cluster complexes need to be protected from interfering molecules by isolating the metallic cluster complexes. Given this need, the present invention describes one approach to achieve this desired result.

### SUMMARY OF THE INVENTION

The present invention provides a luminescent metallic cluster particle that is made up of a luminescent core structure and a material shell, which covers or encapsulates the luminescent core structure. The luminescent core is composed of one or more aggregates of least one or more kinds of luminescent metallic cluster complexes. The aggregate can have either a crystalline or amorphous structure or a combination of both. The shell is comprised of at least a layer of a material film or coating, which provides protection for the core structure and permits one to create new properties for the particle. To such an end, the surface of the shell may be modified or functionalized with a number of chemical functional groups that can serve in further modification of the particle to suit desired uses. The composition of the shell can be small molecules, polymers, hybrid materials, or their combinations. One or more portions of the material shell may be physically or covalently attached to the surface of the core structure.

With respect to the core and shell, according to the invention, it is desired to have a relatively thin film or coating selected from either an organic or inorganic material around at least part of the cluster complexes. By doping or encapsulating luminescent metallic cluster complexes in a polymer shell, for instance, one can also orient the metallic clusters for effective excitation states and greater emissive states.

The present invention also provides applications of the luminescent particle in a variety of fields, particularly in molecular detections, organism detections and imaging. The luminescent particle acts as a label that can be quantified by optical means. One embodiment of the invention is a luminescent particle that has a crystalline core structure formed from a luminescent gold-silver cluster complex and a polymer shell that forms a thin film on the surface of the crystalline core particle. The present luminescent particle has a luminescent metallic cluster core, a thin film shell and biological functional moieties on the surface of the shell. The polymer shell further has surface functional groups which allows further surface functionization of the particles with biological molecules. The luminescent particles with biological molecules can provide a biological label to detect and quantify an analyte. The biological functional moieties are attached to the surface of the shell either covalently or physically. A shell of biocompatiable material can also make the organometallic compounds more compatible with biological systems.

Additional features and advantages of the present invention will be revealed in the following detailed description. Both the foregoing summary and the following detailed description and examples are merely representative of the invention, and are intended to provide an overview for understanding the invention as claimed.

### BRIEF DESCRIPTION OF FIGURES

FIG. 1 is a schematic representation of a core, a, constituted from metallic clusters that may be incorporated in the present invention.

FIG. 2 is a schematic representation, according to the present invention, of a metallic cluster core, a, as shown in FIG. 1, and a polymer material coating, b, which envelopes the metallic cluster core.

FIG. 3 is a schematic representation of a metallic cluster core, a, encapsulated within a polymer material coating, b, as in FIG. 2, and the surface of the polymer coating is functionalized or modified with a chemical functional group, c.

FIG. 4 is a schematic presentation of a metallic cluster core, a, encapsulated within a polymer material coating, b, that has been functionalized with chemical functional groups, c, adapted to attract or bind a biological moiety, d.

FIG. 5 is an excitation (excited at 350 nm) spectrum of an Au(I)—Ag(I) cluster, showing an intensity peak at about 360 nm.

FIG. 6 is an emission spectrum of the Au(I)—Ag(I) cluster, showing an intensity peak at about 450 nm.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the”, include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

Metal complexes suitable for the present invention can be soluble in various solvents such as ethers, halogenated solvents, hydrocarbons, etc. In some embodiments of the invention, the metal complexes can be employed at its solid state form. The term “solid state” shall mean that the metal complex may be in a crystalline form or an amorphous form. The term “crystalline” shall mean that the metal complex assumes an ordered packing order which may be defined by a certain parameters such as a crystal cell dimensions, a space group, etc. In some embodiments of the invention, the “crystalline” solid states are critical to desired luminescence properties because of potential metal-to-metal interactions between discrete molecules. The term “aggregate” refers to at least two molecules of metallic clusters coordinated or arranged together to form a solid structure.

The term “cluster,” “metal cluster,” or “metallic cluster complex” refers to an organometallic structure that contains at least three metal centers with associated ligand(s). The three metal centers may be of either the same or different kinds of metal atoms. The metal centers may or may not have direct bonds with each other, depending upon the structure and bonding nature of the clusters between ligands and metal centers. Metallic cluster complexes can assume different structures. The structural types, if only metal centers are considered, may include linear, triangle, tetrahedron, butterfly, pentagon, octahedron, etc. These structures may be symmetrical or asymmetrical if the complex be viewed in respected to a plane or a center.

The term “homonuclear” refers to a metallic cluster complex that contains only one type of metal atom.

The term “heteronuclear” or “mixed metallic cluster complex” (or clusters) refers to a metal complex that contains more than one type of metal atom.

The term “mononuclear” metal complex refers to a metal complex that has only a single metal center with associated ligand(s).

The term “polynuclear” metallic cluster complex refers to a metallic cluster complex that has at least three metal centers with associated ligand(s). The metal centers may be either of the same or different kinds of metal atoms.

The term “ligand” as used herein refers to a molecule, ion, or atom that is attached to a central atom of a coordination compound, chelate, or other complex.

The term “chelating ligand,” as used herein, refers to a ligand that can form a ring around or includes a metal atom. A chelating ligand typically has at least two attachment sites for the same metal atom or different metal atoms.

The term “monodentate ligand,” as used herein, refers to ligand species that have only one point of attachment to a metal center, as distinct from a “polydentate ligand,” which has more than one. Suitable ligands for the present invention may be selected from any group of ligands that can form a luminescent metal complex; such ligands may include: phosphines, amines, acids, alcohols, thiols, etc. In some embodiments, a combination of different groups (e.g., phosphines and amines or others) can be in the same ligand. In some embodiments, additional functional groups can be attached to these ligands to enable one to fine-tune the luminescence properties of a given metal complex series.

The prefixes “mono-, di-, tri, and tetra-” indicate the number of occurrences of a ligand in a given organometallic complex. For example, if a complex has four triphenyl phosphine ligands, it can be described as tetra(triphenylphosphine) complex. Similarly, the prefixes “mono-, di-, tri, and tetra-” also can indicate the number of occurrences of a metal atom in a given complex. For example, if a complex has three cobalt atoms, the complex can be referred to as a tricobalt complex.

The term “binder” or “marker” refers to a biological, chemical, or biochemical molecule that can recognize and bind with a particular membrane protein. The binder or marker can be a ligand, a protein, an antibody, or an aptamer (e.g., DNA-, RNA-, or peptide-aptamers). When the binder is a protein, an antibody that can bind with the protein may be used as a readout molecule, preferably, in a secondary or sequential step. The binder or marker can be either labeled or unlabeled. If labeled, the label can be any of the following: a fluorescent tag, a radio-isotope, a nano-particle (e.g., gold particle, quantum dots, etc.), or biotin.

The term “biological molecule” or “biomolecule” refers to any kind of biological entity, including, such as modified or unmodified nucleotides, nucleosides, peptides, polypeptides, proteins, protein domains, fusion proteins, antibodies, membrane proteins, lipids, lipid membranes, cellular membranes, cell lysate, oligosaccharides or polysaccharides, cellulose, carbohydrates, heptins, or lectins.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon.

The term “aggregation” is a general term that describes the tendency of large molecules or colloidal particles to combine in clumps or clusters.

The term “doping” or “encapsulation” as used herein refers to either impregnating or enveloping a luminescent organometallic complex within a polymer material, for example, either by direct precipitation within the polymer or covering a luminescent metallic cluster complex coated surface with a polymer layer.

The process of encapsulation, according to certain embodiment, may involve using a solution of a solid solute (such as a solid luminescent metallic cluster complex aggregate) in a solution of encapsulating material (e.g., an organic polymer matrix, or metallic film). Such encapsulation may be generated by creating a liquid suspension containing luminescent cluster aggregates and a liquid solution of a polymer and then co-precipitating the luminescent metallic cluster aggregates and polymer so that the cluster aggregates would be dispersed inside a matrix of the solid polymer material. The particles thus precipitated may be viewed as a solid solution of a luminescent metallic cluster aggregates dispersed in a solid polymer matrix. Other techniques may be employed to form a shell of inorganic silicate or silicon, or metallic film layer.

### Section II—Description

The present invention provides a luminescent metallic cluster particle that contains at least one type of luminescent metal complex or cluster, forming a core structure, and a material shell that covers the core structure. The luminescent core structure has one or more aggregates of organometallic complex molecules. In certain embodiments, the metallic cluster complexes are aggregated together and encapsulated or doped within a polymer material matrix. The metallic cluster can have at least three metal atoms forming a single center or core of either a mononuclear, binuclear, or polynuclear conformation with either homo- or hetero-species of metal atoms situated within a ligand structure. The metallic cluster molecules can be crystalline or amorphous structure. The metallic cluster molecules can have or can be arranged desirably in a crystalline structure or phase to enhance the luminescent properties of the metal cluster. The ligands also can comprise a variety of organometallic configurations.

The shell that overlays the core may comprises one or more layers of thin films formed predominately of organic polymers, inorganic polymers, hydrid polymers, or combinations thereof. Each layer of the thin films can be either a monolayer or multiple layers. The different embodiments of the present invention have metal complexes that are surrounded by a protective polymer shell or coating. Alternatively, the metallic cluster complexes may be described as being doped within the polymer material matrix.

The polymer material encapsulates a metal complex in a polymeric matrix that enables the metallic cluster complex to be attached as labels or used in certain assays. Since naked molecules of the metallic cluster complexes of interest typically doe not have proper sites that can allow one to attach with biological molecules, one aspect of the present invention is to modify the metallic cluster complexes and make them compatible to use as labels in various assays. The film that overlays the organometallic molecules will provide a functionalized surface for attaching biological molecules. The present invention envisions a new use for organometallic molecules that have been typically toxic to biological systems; hence, not useful for biological assays. The invention involves adapting or encapsulating an aggregate of least two organometallic cluster molecules, preferably with a crystalline structure, in a polymer material matrix to make the organometallic clusters compatible with biological systems for use as a detection probe or marker in biological or biochemical assays.

In some embodiments, the luminescent metallic cluster complex has at least three metal nuclei, with or without direct bonding between each of the metal nuclei. The metal nuclei can host either an identical metal element or different metal elements. The aggregates can have two or more metallic cluster complex molecules stacked together. In some examples, the metallic cluster complex can includes Au(I)—Ag(I) polynuclei, among others.

Recent industrial interest in luminescent metallic cluster complexes has been driven by a variety of potential applications. Scientists have reported recently the discovery of a new class of isostructural, brilliantly luminescent gold-silver cluster complexes (Wang et al., J. Am. Chem. Soc., 2004, 126, 9488-9489). The luminescence spectra of this new series of metallic cluster complexes can be tuned by simply changing the constituent of the R-groups or side chains of the ligand. The gold-silver complexes have a μ₃-bridging atom selected from Group 16 of the Periodic Table, in which the emission energy changes strikingly from blue to yellow to orange in going from O to S to Se. This observation is a first report of luminescence from a gold(I) oxo system. The Au(I)—Ag(I) cluster complexes have a general formula: [Au₃(μ₃-E)Ag—(PPh₂py)₃]X₂, wherein E=O, S, Se, PPh₂py=diphenylphosphine-2-pyridine, and X=negatively charged counter ions, such as BF₄, BF₆, or PF₆. A few potential uses have been proposed for these complexes. The gold-silver cluster complexes, however, have several drawbacks for many applications. For instance, the complexes are not compatible with aqueous and biological systems. This aqueous incompatibility prevents the complexes from being used directly for biological detection and imaging. Moreover, since it is believed that the nature of the luminescence is phosphorescence, the complexes can be susceptible to easy quenching from exposure to oxygen, nitrogen oxide, or other oxidative or reactive molecules.

The luminescent particles can employ a variety of different kinds of metal cluster complexes. In addition to Au(I)—Ag(I) cluster complexes, other luminescent materials or molecules may also be doped within each particle matrix. For instance, the luminescent particle can have luminescent metal clusters with homo-metal species, such as described by Che et al., in Polyhedron, Vol. 13, No. 6/7, pp. 887-890, 1994. Alternatively, luminescent heteronuclear metal clusters, such as described by Wei et al., in J. Am. Chem. Soc. 2004, 126, 9940-9941, can be used to dope the present luminescent particles, as long as at least two molecules of the metal cluster complex are oriented in close spatial proximity to each other. Other luminescent metal clusters that can be used to dope the present material particles include various examples described in the scientific literature, such as Turk et al., “Molecular Models for Semiconductor Particles,” Photosentitive Metal-Organic Systems, pp. 233-241, 1993; James et al., “Phosphorescence and Structure of a Tetrameric Copper (I)-Amide Cluster,” Inorg. Chem. 1998, 37, 3785-3791; Gray et al., “Highly Emissive Hexanuclear Rhenium (III) Clusters Containing the Cubic Cores [Re₆S₈]²⁺ and [Re₆Se₈]²⁺,” Inorg. Chem. 1999, 38, 5932-5933; Wei et al., “Luminescent Ag(I)—Cu(I) Heterometallic Hexa-, Octa-, and Hexadecanuclear Alkynyl Complexes,” Inorg. Chem. 2004, 43, 3484-3491; Yam et al., “Design of Luminescent Polynclear Cu(I) and Ag(I) Complexes with Chalcogenide and Acetylides as Bridging Ligands,” Coordination Chemistry Reviews, 171 (1998) 17-41; Ara et al., “Synthesis, Structures and Photophysics of Novel Luminescent Platinium-Copper Complexes,” J. Organometallic Chem., 670 (2003) 221-234; Pierre D. Harvey, “Luminescence Properties of Organometallic/ Coordination Oligomers and Polymers Containing Diphosphine and Diisocyanide Assembling Ligands: Comparison Between Mononuclear Model Complexes and Polymers,” Macromol. Symp. 2004, 209, 81-95. The contents of all of the preceding articles or papers, respectively, are incorporated herein by reference.

The material used to create the luminescent particle can include a variety of polymers, co-polymers, block copolymers, at least partially cross-linked or non-cross-linked polymers, and their mixtures, which are attached to said metallic cluster complex core. Desirably, the polymer is surface functionalized and may include polymeric surfactants or polyelectrolytes.

The particle may further incorporate at least a second type of luminescent molecule. The second luminescent molecules can be one of the following: a different metallic cluster complex, an organic fluorescent molecule, an inorganic luminescent molecules, phosphorescent molecules, and quantum dots.

The organometallic cluster complex and the second of luminescent molecule can either exhibit luminescence independently of each other, or may depend upon one another to generate luminescent spectral properties through fluorescence resonant energy transfer. In the latter case, the second luminescent molecule can depend on an excitation from said metallic cluster complex to produce a luminescent emission; or, the metallic cluster complex can depend on an excitation from second luminescent molecule to generate its own luminescence.

The polymer material matrix can have a number of functional groups incorporated as part of the matrix for creating a particle with a functionalized exterior surface. The functional groups may include at least one of the following: an amine, an alcohol, an imide, a carboxylic acid, a sulfate or sulfide, an aldehyde, an epoxy, or a combination thereof. The metal cluster complexes exhibit a high density of surface charges, of greater than or equal to about 5-10/nm² or about 10,000/Å². The material particles can have a particle size that ranges from about 5 or 10 nm to about 20 or 30 μm. According to certain desired embodiments, the mean particle size may range from about 20 nm to about 10 μm, more desirably from about 30 nm to about 900 or 1000 nm. Depending on the particular application, typical size ranges are from about 40 nm or 50 nm up to about 700 nm or 800 nm, or any combination or variation in between. The particles can be either a solid or semi-solid, or porous material (e.g., micro- or nano-scale pores).

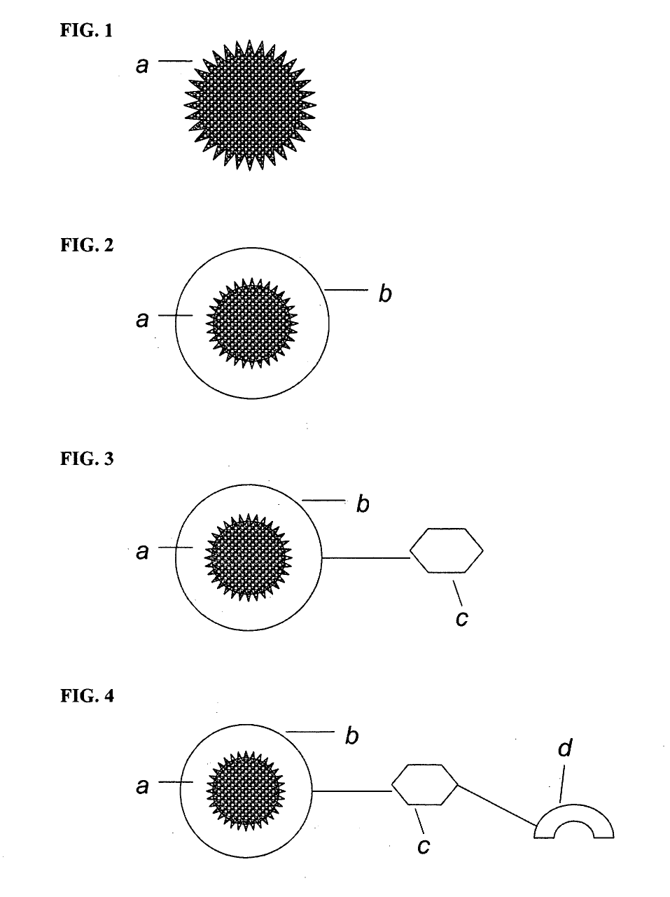

FIG. 1, shows a schematic, macro-representation of a luminescent core, a, which can be incorporated into the present luminescent particle. The core is composed of one or more aggregates of at least one of kind of luminescent metal cluster complex. The metal cluster complex may be either a homonuclear or heteronuclear species, and include various kinds of metal atoms, coordinated with ligands that form oligomers and polymers of mixed-bridging organometallic complexes. The metal atoms in the cluster coordination may include: Al, Ag, Au, Cd, Ce, Co, Cr, Cu, Hg, Fe, Ir, In, Mn, Mg, Mo, Ru, Fe, Ni, Os, Re, Mo, W, Sn, Zn, Pt, Pd, Ta, Nb, V, Ti, Zr, Sc, Re, Te, Y, or an alkaline rare earth element, such as: La, Ce, Pr, Nd, Pm, Sm, Eu, Gd, Tb, Dy, Ho, Er, Tm, Yb or Lu. Preferably, the rare earth element is Er, Nd, Pr, Eu, Dy or Tm. More preferably, the rare earth element is Er. In certain desired embodiments, silver (I), gold (I), copper (I), palladium (I), or platinum (I) are arranged in chelating or bridging ligand structures.

The ligands may be a molecule, ion, or atom that is attached to a central atom of a coordination compound, chelate, or other complex. The chelating ligands may form a ring around or includes a metal atom. A chelating ligand typically has at least two attachment sites for the same metal atom or different metal atoms. The chelating ligands may have only called “monodentate ligand”, or more than one point of attachment to a metal center called “polydentate ligand”.

Suitable ligands for the present invention may be selected from any group of ligands that can form a luminescent metal cluster complex; such ligands may include: phosphines and their derivatives, porphyrins and porphine and its derivatives, polypyridyl and its derivatives, pyridine, pyrazine, isonicotinamide, imidazole, bipyridine, terpyridine, phenanthroline and dipyridophenazine, amines, acids, alcohols, thiols, etc. In some embodiments, a combination of different groups (e.g., phosphines and amines or others) can be in the same ligand. In some embodiments, the bridging ligands can be any one of the following but not limited to: Diphenyl-2-pyridyl-phosphine (DPPY). 1,8-diisocyano-p-menthane (DMB), bis(diphenylphosphino) menthane (DPPM), -ethane (DPPE), -propane (DPPP), -butane (DPPB), -pentane (DPPPEN) and -hexane (DPPH), bis(diphenylphosphino) acetylene(DPA), bis(dimethylphosphino) menthane (DMPM). Suitable ligands may be substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide. In some embodiments, additional functional groups can be attached to these ligands to enable one to fine-tune the luminescence properties of a given metal complex series.

Not to be bound by theory, it is believed that ligands absorb incident photo energy within the metal cluster. As the energy state of the metal nuclei is excited, the ligands channel the energy states. The ligands are also self-shelding in an aggregate conformation once in a crystalline orientation. Changes in the orientation or structure of ligands or ligand constituents can influence and change the energy state of the metal clusters when under photo-induced excitation and ultimate manifestation of luminescence.

The metal cluster complex can be soluble in various solvents such as ethers, halogenated solvents, hydrocarbons, etc. The metal cluster complexes can be employed at its solid state form: crystalline form or amorphous form. In crystalline form, the metal complex assumes an ordered packing order which may be defined by a certain parameters such as a crystal cell dimensions, a space group, etc. In some embodiments of the invention, the “crystalline” solid states are critical to desired luminescence properties because of potential metal-to-metal interactions between discrete molecules. The metal centers in the metal cluster complex may or may not have direct bonds with each other, depending upon the structure and bonding nature of the clusters between ligands and metal centers. The metal cluster complexes can have different structures. The structural types, if only metal centers are considered, may include linear, triangle, tetrahedron, butterfly, pentagon, octahedron, etc. These structures may be symmetrical or asymmetrical if the complex be viewed in respected to a plane or a center.

The metal centers in the metal cluster complex may have only one type of metal element (i.e., “homonuclear”,) or more than one type of metal element (i.e., “heteronuclear”,). Also, the metal centers in the metal cluster complex may have at least three metal atoms forming a single center or core (i.e., “mononuclear”,) with associated ligand(s), or have two metal centers (i.e., “binuclear”,) with associated ligand(s), or have more than three metal centers (i.e., “polynuclear”,) with associated ligand(s). The metal centers may be composed of either the same or different kinds of metal atoms.

FIG. 2, illustrates an exemplar embodiment of luminescent particle core (a) and shell (b) structure. The shell (b) can have one or more layers of thin films formed predominately of organic polymers, inorganic polymers, hydrid polymers, or combinations thereof. Each layer of the thin films can be either a monolayer or multiple layers.

The shell (b) may include, is but not limited to, small molecules, inorganic materials, hybrid materials, oligomers and polymers. The polymers may include but limited to homopolymers, co-polymers, block copolymers, un-crosslinked polymers, partially cross-linked polymers, cross-linked polymers, amphiphilic polymer, polymeric surfactants, polyelectrolytes, and their mixtures, etc. In particular, the polymers and copolymers may include, but not limited to, polystyrene, polyacrylonitrile, polyethylacrylate, polybutylacrylate, polyalkyl-acrylate, polymethacrylic acid, polyvinyl fluoride, polyvinyl chloride, polyvinyl bromide, polyvinyl iodide, polyvinylidene fluoride, polyvinylidene chloride, polyvinylidene bromide, polyvinylidene iodide, their derivatives and combinations thereof. The binding between core (a) and shell (b) in a luminescent particle structure may be the result of, but not limited to, physical aggregation, or covalent, ionic, and other chemical binding.

FIG. 3, illustrates an exemplar embodiment of luminescent particle comprising core (a) shell (b) and exterior surface functional groups (c). The exterior surface functional groups (C) include at least one of the following: an amine, an alcohol, an imide, a carboxylic acid, a sulfate or sulfide, an aldehyde, an epoxy, or a combination thereof.

The exterior surface functional groups (c) may be achieved by selecting shell (b) polymers with functional monomers from, but not limited to: (methyl)acrylic acid, crotonic acid, itaconic acid, and other unsaturated carboxylic acids; sodium styrenesulfonic acid and other unsaturated sulfonic acid salts; 2-hydroxyethyl (methyl)acrylate, glycidyl (methyl)acrylate and other (methyl)acrylate esters; and (methyl)acrylamide and N-methylol(methyl)acrylamide. The shell (b) materials may also be composed of vinyl monomers incorporated with crosslinkable monomers, if desired, to provide stability and improve resistance to blocking, heat and solvent. The crosslinkable monomers which can be used include, for example, carboxylic acid, ethanolamine, amine, amino, imine, isocyanate, melamine, metal alkoxides, monomer with hydroxyl, epoxy and hydrolysable organosilyl groups, beta-ketoester, ethylene glycol, divinylbenzene, ethylene glycol di(methyl)acrylate, trimethylolpropane trimethacrylate and other monomers. The exterior surface functional groups (c) may also achieve through other chemical of oxidation process like plasma treatment and oxidation treatment.

The luminescent particle should contain an aggregate of at least two metal cluster molecules. Desirably, the metal clusters are arranged in a crystalline structure relative to each other. The present tunable metal compounds, once encapsulated within a protective polymer casing can be applied to a variety of uses. For instance, the metal-doped polymer particles can be used to perform applications similar to those that presently use quantum dots. One can employ the metal-doped polymer particles for detection in biological or biochemical assays or processes. Since each of the polymer particles has a surface that can be modified chemically (i.e., functionlizable) or adapted to promote stable attachment to a desired or predetermined substrate, such as a nature or synthetic moiety or molecule, biological or non-biological polymer surfaces, cellular membranes, organelles, nucleic acids, and small or micro-molecules such as amino acids, peptides, proteins, steroids, cholesterol, hormones, or antigens, the polymer particles can be readily used to monitor for change in photo luminescent signal in terms of color or intensity.

In assays the ultra-sensitive luminescent metal cluster complex particles can be employed for detection of biological molecules and species. FIG. 4, illustrates an exemplar embodiment of the luminescent particle tagging a biological moiety (d) on with exterior functional surface. The biological moiety (d) is attached to the exterior surface through covalent or non-covalent bonds. By associating the functionalized exterior surface of the particle to tag a biological moiety, a luminescent particle can be used in biological assays or detection. The biological moiety (d) may include antibodies, antigens, hormone molecules, nucleic acids, modified or unmodified nucleotides, nucleosides, peptides, polypeptides, proteins, protein domains, fusion proteins, antibodies, membrane proteins, lipids, lipid membranes, cellular membranes, cell lysate, oligosaccharides or polysaccharides, cellulose, carbohydrates, heptins, or lectins.

The biological moiety (d) can be attached by means of any stable physical or chemical association, to the surface functional group of the luminescent metal cluster complex particles directly or indirectly by any suitable means. The biological moiety (d) is attached to the exterior surface according to either covalent or non-covalent bonds. Desirably, the biological moiety is attached to the attachment group directly or indirectly through one or more covalent bonds. If the biological moiety (d) is attached to the surface functional group indirectly, the attachment preferably is by means of a linker. The term “linker,” as used herein encompasses any suitable means that can be used to link the biological moiety (d) to the surface functional group of the luminescent metal cluster complex particles. If the conjugate is to be used in vivo, biologically compatible linker would be desirable.

For example, if the attachment group is mercaptoacetic acid and a nucleic acid biomolecule is being attached to the attachment group, the linker preferably is a primary amine, a thiol, streptavidin, neutravidin, biotin, or a like molecule. If the attachment group is mercaptoacetic acid and a protein biomolecule or a fragment thereof is being attached to the attachment group, the linker preferably is strepavidin, neutravidin, biotin, or a like molecule. In accordance with the invention, the linker should not contact the protein biomolecule or a fragment thereof at an amino acid which is essential to the function or activity of the attached protein. Crosslinkers, such as intermediate crosslinkers, can be used to attach a biomolecule to the attachment group of the luminescent metal cluster complex particles. Ethyl-3-(dimethylaminopropyl) carbodiimide (EDAC) is an example of an intermediate crosslinker. Other examples of intermediate crosslinkers for use in the present invention are known in the art. See, for example, Bioconjugate Techniques (Academic Press, New York, (1996)). Catalytic crosslinkers also can be used to attach a biomolecule to the attachment group of the luminescent metal cluster complex particles. Catalytic crosslinkers effect direct attachment of the biomolecule to the attachment group. Examples of catalytic crosslinkers are also known in the art. See, for example, Bioconjugate Techniques (1996), supra. Attachment of a biomolecule to the surface functional group of the luminescent metal cluster complex particles also can be effected by a bi-functional compound as is known in the art. See, for example, Bioconjugate Techniques (1996), supra. In those instances where a short linker could cause steric hindrance problems or otherwise affect the functioning of the biomolecule, the length of the linker can be increased, e.g., by the addition of from about a 10 to about a 20 atom spacer, using procedures well-known in the art (see, for example, Bioconjugate Techniques (1996), supra). One possible linker is activated polyethylene glycol, which is hydrophilic and is widely used in preparing labeled oligonucleotides.

According to another aspect, the present material particles may be employed in optical or imaging applications. The optical or imaging application may include an OLED display, solar photoconversion, biological assays, sensing, optical detection and imaging uses. Such uses may involve the luminescent particle having a metallic cluster complex be doped within a polymeric matrix, and the luminescent particle having a functionalized exterior surface adapted to be either applied on or associate with at least a portion of either an inorganic or organic substrate.

### Section III—Fabrication

The present luminescent metal cluster particles can be fabricated according to methods and processes that involve a coating procedure, which includes the steps of: (1) dispersing luminescent metal cluster complex in a solution suspension mixture; (2) mixing the solution suspension mixture with a polymer or co-polymer solution for a period of time to let polymer or co-polymers (shell material) to coat the luminescent metal cluster complex (core material) and form a core/shell structure; (3) separating the core/shell polymer or co-polymer coated luminescent metal cluster complex particles; (4) washing the formed particles; and (5) drying the particles or suspending the particles in storage solvents.

The present invention further provides a method of obtaining luminescent metal cluster complex particles that contain more than one kind of luminescent metal cluster, each of which can exhibit luminescence independently of each other, or may depend upon one another to generate luminescent spectral properties through fluorescence resonant energy transfer. The method may include the steps of: (1) uniformly dispersing more than one kind of different luminescent metal cluster complexes in a solution suspension mixture; (2) mixing the solution suspension mixture with polymers or co-polymers solution for a period of time to let polymers or co-polymers (shell material) adhere or cover onto more than one luminescent metal cluster complexes (core material) to form core-shell structure; (3) separating the core-shell polymer or co-polymer coated luminescent metal cluster complex particles; (4) washing the formed particles; and (5) drying the particles or suspending the particles in storage solvents.

The present invention further provides a method of making luminescent metal cluster complex particles that include a non-metallic cluster luminescent molecule which can either exhibit luminescence independent of each other, or may depend upon one another to generate luminescent spectral properties through fluorescence resonant energy transfer. The method comprises include the steps of: (1) dissolving a luminescent molecule in a dissolving polar (e.g., aqueous) solvent to form a solution I; (2) dispersing luminescent metal cluster complex in solution I to form a luminescent metal cluster complex/luminescent molecule suspension mixture II; (3) mixing the suspension mixture II with polymers or co-polymers solution to let polymers or co-polymers (shell material) cover the luminescent metal cluster complex/luminescent molecule aggregate (core material) to form a core-shell structure; (4) separating the polymer or co-polymer coated luminescent metal cluster complex/luminescent molecule particles; (5) washing the formed particles; and (6) drying the particles or suspending the particles in storage solvents.

### EXAMPLES

The present invention is described further in the following examples. These examples serve to illustrate further the present invention and are not intended to limit the scope of the invention.

Materials:

Diphenyl-2-pyridyl-phosphine (dppy)

Gold (I) Chloride\Sodium Tetrafluoroborate

Dichloromethane

Diethyl ether

Silver Nitrate

Sodium Hydroxide

All chemicals were purchased from Aldrich and used as received.

According to the synthesis procedure described by Wang et al., J. Am. Chem. Soc., 2004, 126, 9488-9489, we proceeded to produce in Examples 1 a gold(I)-Diphenyl-2-pyridyl-phosphine (Au(dppy)Cl) intermediate, and in Example 2, a gold(I)-Silver(I) Cluster using the Au(dppy)Cl intermediate.

### Example 1

Using about 0.44 g of Diphenyl-2-pyridyl-phosphine (dppy) was dissolved 20 ml Dichloromethane Solution and was drop-wisely added into 60 ml Gold (I) Chloride (0.5 g) Dichloromethane Solution while stirring for 4 hours. The reactant solution was then filtered, and 400 mL of Hexane was then added into the resultant dichloromethane solution. The participates were collected and was vacuum dried for one hour before next synthesis step. The ³¹P-NMR spectrum for the intermediate in CD₂Cl₂ is about 32.66 ppm.

### Example 2

About 0.3 g Au(dppy)Cl, 0.3 g Ag2O (freshly prepared from AgNO3 and NaOH) and 0.40 g NaBF4 were suspended in 50 ml acetone/water solution (acetone: water volume ratio=4:1). After the reaction mixture was stirred for 3 hours, acetone was removed under vacuum. The ³¹P-NMR spectrum for the Au(I-Ag(I) cluster in CD₂Cl₂ is about 28.06 ppm. The residue was extract with dichloromethane (3×20 ml) and the volume of the resultant dichloromethane solution was reduced to around 10 mL. Light-brown crystal like metal cluster was formed after layering with diethyl ether. The metal cluster was dried under vacuum for one hour.

### Example 3

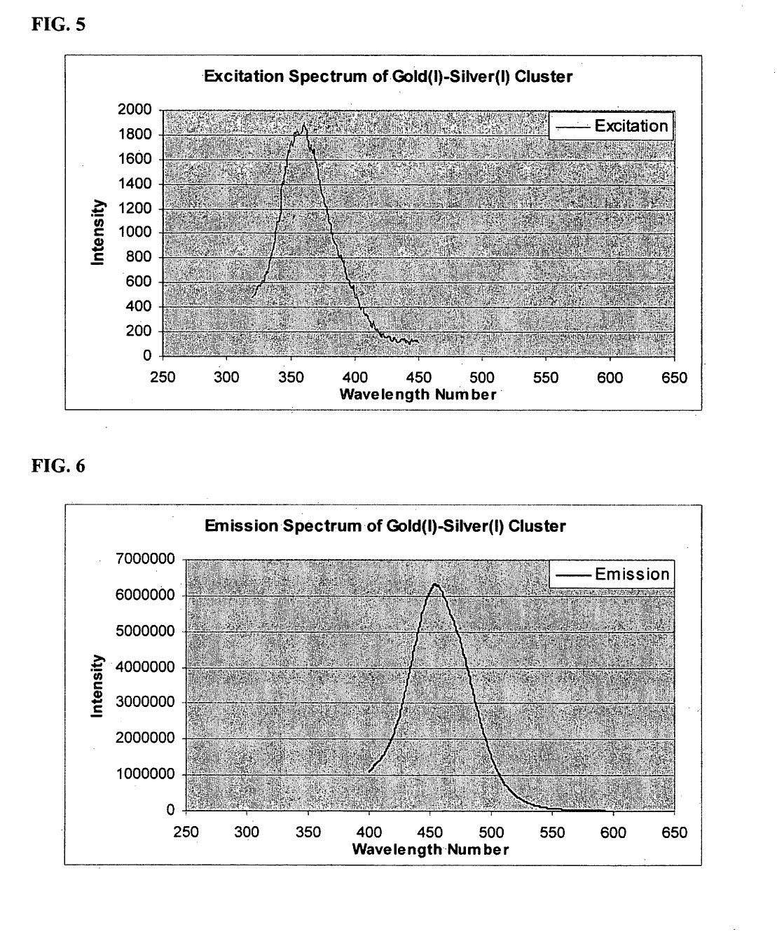

About 5 mg of Gold(I)-Silver(I) Cluster made in Example 2 was dispersed in 1 mL D.I. water by probe sonicating for 5 minutes. After setting at still condition for five minutes, top 100 uL of suspension was diluted with 500 μl distilled water for measurement of phosphorescent intensity. The instrument used was a Fluorolog SPEX 1934D phosphorimeter (from JoBin Yvon Horiba, Edison, N.J.) The parameters used in the measurements were; excitation light at 350 nm, phosphorescence at 450 nm, number of scans was 1, initial delay was 0.01 ms, the sample window was 3 ms, the time per flash was 50 ms and the number of flashes was 20. FIG. 5 shows an excitation (excited at 350 nm) spectrum of the Au(I)—Ag(I) cluster with an intensity peak at about 360 μm, and FIG. 6 showed the emission spectrum of the Au(I)—Ag(I) cluster with an intensity peak at about 450 nm.

### Example 4

We proceeded to fabricate luminescent metal cluster particles using Gold(I)-Silver(I) Cluster as a core arid a amphiphilic polymer (surfactant) polymer as a protective coating layer: About 5 mg of Gold(I)-Silver(I) Cluster made in Example 2 was dispersed in 1 mL D.I. water by probe sonicating for 5 minutes. After setting at still condition for five minutes, top 200 μL of suspension was transferred into two vials with 100 μL suspension into each vial with 500 μL D.I water inside. 20 μL of Polyvinyl Sulfonic acid (PVS) was added into one vial and 20 μL of D.I water was added into the second vial as control. Both vials were then slowly rotated for one hour.

### Example 5

We washed the polymer coated Au(I)—Ag(I) luminescent metal cluster particles. The polymer coated Au(I)—Ag(I) luminescent metal cluster particles made in Example 5 along with uncoated control particle suspension were placed into 1.5 ml vials and centrifuged at 14,000 rpm for 20 minutes. The supernatant was discarded and a volume of water equal to that discarded was added to each vial. The vials were placed in an ultrasonic bath for 5 minutes to re-suspend the particles. The particles' zeta potential results were measured (80 ul particle suspension into 3.0 mL 1 mM KCl) and summarized in Table 1.

TABLE 1
Zeta Potential Results of Polyvinyl Sulfonic acid Polymer
(PVS) Coated Gold(I)-Silver(I) and Uncoated Gold(I)-Silver(I)
Luminescent Metal Cluster Particles.

| | Run 1 | Run 2 | Run 3 | Mean | Std. |
|---|---|---|---|---|---|
| | (mV) | (mV) | (mV) | (mV) | Dev. |
| Uncoated | 6.48 | 4.44 | 3.02 | 4.65 | 1.00 |
| Gold(I)- | | | | | |
| Silver(I)Particle | | | | | |
| PVS coated | −34.06 | −30.11 | −35.57 | −33.25 | 1.60 |
| Gold(I)- | | | | | |
| Silver(I)Particle | | | | | |

### Section IV—Uses & Applications

The present invention has application in various diagnostic assays, including, but not limited to, the detection of bacterial infection, yeast infection, viral infection, parasite infection, cancer, cardiac disease, liver disease, genetic diseases, and immunological diseases.

The present invention provides a method of detecting a nucleic acid in a sample. The method comprises attaching a nucleic acid capture probe to a solid support. The nucleic acid capture probe comprises a sequence that binds to the nucleic acid in the sample. The attached nucleic acid capture probe is then contacted with the sample, thereby immobilizing the nucleic acid on the solid support. The method further comprises contacting the immobilized nucleic acid with a conjugate comprising luminescent metal cluster complex particles and a biomolecule. The biomolecule of the conjugate specifically binds to the nucleic acid. Then, the method comprises detecting luminescence. The detection of luminescence indicates that the conjugate bound to the nucleic acid in the sample.

The present invention also provides a method whereby two or more different molecules and/or two or more regions on a given molecule can be simultaneously detected in a sample. The method involves using a set of conjugates as described above, wherein each of the conjugates in the set has a different luminescent metal cluster complex in the particle attached to a biomolecule that specifically binds to a different molecule or a different region on a given molecule in the sample. Preferably, the luminescent metal cluster complex particles of the conjugates range in size from 10 nm to 500 nm. The luminescent metal cluster complex particles that corresponds to a particular color emission is well-known in the art. Any variation of composition between luminescent metal cluster complex particles can be used as long as the luminescent metal cluster complex particles differing in composition can be excited at a single wavelength and differences in the luminescence between the luminescent metal cluster complex particles of different composition can be detected.

The present invention can be used in a diagnostic assay to detect certain viruses, such as HIV and hepatitis, by, for example, (a) removing a sample to be tested from a patient; (b) contacting the sample with a infection biomolecular luminescent metal cluster complex particles conjugate, wherein the biomolecule is an antibody or antigenically reactive fragment thereof that binds to the virus; and (c) detecting the luminescence, wherein the detection of luminescence indicates that the virus is present in the sample. The patient sample can be a bodily fluid, such as saliva, tears, blood, serum or urine.

The present invention also can be used in a diagnostic assay to determine ultra-low-level viral loads of certain viruses, such as HIV and Hepatitis, by detecting the viral nucleic acid. Determining the viral load of a patient is useful in instances where the number of viral particles is below the detection limits of current techniques. For example, this technique can be particularly useful for tracking ultra-low HIV levels in AIDS patients during advanced drug treatment, such as triple drug therapy, in which the viral load of the patient has been greatly reduced. The detection of viral nucleic acid can be accomplished by, for example, (a) removing a sample to be tested from a patient; (b) treating the sample to release the viral DNA or RNA; (c) contacting the sample with luminescent metal cluster complex particles biomolecular conjugate, wherein the biomolecule binds to the nucleic acid of the virus; and (d) detecting the luminescence, wherein the detection of luminescence indicates that the virus is present in the sample.

One embodiment of the inventive method of detection of viral nucleic acid is accomplished by (a) removing a sample to be tested from a patient; (b) treating the sample to release the viral DNA or RNA; (c) attaching capture probes to a solid support, wherein the capture probes comprise a sequence that binds to the viral nucleic acid in the sample; (d) contacting the attached capture probes with the viral nucleic acid, thereby immobilizing the viral nucleic acid on the solid support; (e) contacting the immobilized viral nucleic acid with luminescent metal cluster complex particles conjugate, wherein the biomolecule of the conjugate specifically binds to the viral nucleic acid; and (f) detecting luminescence, wherein the detection of luminescence indicates that the conjugate bound to the viral nucleic acid in the sample.

The present invention also can be used to detect a disease state, such as a genetic disease or cancer, by (a) removing a sample to be tested from a patient; (b) contacting the sample with luminescent metal cluster complex particles biomolecular conjugate, wherein the biomolecule is a nucleic acid that specifically hybridizes with a nucleic acid of interest; and (c) detecting the luminescence, wherein the detection of luminescence indicates the existence of a given disease state. In these cases, the sample can be a derived from a cell, tissue or bodily fluid. The gene of interest can be a marker for a disease-state, such as BRCAI, which may indicate the presence of breast cancer.

The above-described methods also can be adapted for in vivo testing in an animal. The conjugate should be administered to the animal in a biologically acceptable carrier. The route of administration should be one that achieves contact between the conjugate and the biomolecule, e.g., protein or nucleic acid, to be assayed. The in vivo applications are limited only by the means of detecting luminescence. In other words, the site of contact between the conjugate and the biomolecule to be assayed must be accessible by a luminescence detection means. In this regard, fiber optics can be used. Fiber optics enable light emission and detection as needed in the context of the present inventive methods.

In another embodiment, the present invention also provides a composition comprising luminescent metal cluster complex particles as described above and an aqueous carrier. Any suitable aqueous carrier can be used in the composition. Desirably, the carrier renders the composition stable at a desired temperature, such as room temperature, and is of an approximately neutral pH. Examples of suitable aqueous carriers are known to those of ordinary skill in the art and include saline solution and phosphate-buffered saline solution (PBS).

The present invention has been described in general and in detail by way of examples. Persons skilled in the art understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

We claim:

1. A material particle comprising a luminescent metallic cluster complex constituted of at least two metal cluster molecules, forming a core encapsulated within a protective polymer coating.

2. The material particle according to claim 1, wherein said metallic cluster complex has at least three metal atoms.

3. The material particles according to claim 1, wherein said metal cluster includes a single species of metal atoms and ligands.

4. The material particles according to claim 1, wherein said metal clusters include two or more different species of metal atoms and ligands, which chelate in two or more different ways.

5. The material particles according to claim 1, wherein said metal atoms include any one or a combination of the following: Al, Ag, Au, Cd, Ce, Co, Cr, Cu, Hg, Fe, Ir, In, Mn, Mg, Mo, Ru, Fe, Ni, Os, Re, Mo, W, Sn, Zn, Pt, Pd, Ta, Nb, V, Ti, Zr, Sc, Re, Te, Y, or an alkaline rare earth element.

6. The material particle according to claim 5, wherein said rare earth element includes: Er, Nd, Pr, or Tm.

7. The according to claim 1, wherein said metal cluster complex includes Au-Ag cluster complexes that have a general formula: [Au₃(μ₃-E)Ag—(PPh₂py)₃]X₂, wherein E=O, S, Se, PPh₂py=diphenylphosphine-2-pyridine, and X=negatively charged counter ions.

8. The material particle according to claim 1, wherein an aggregate has two or more metal cluster complexes together.

9. The material particle according to claim 1, wherein said aggregate of metallic clusters has an amorphous structure.

10. The material particle according to claim 1, wherein said aggregate of metallic clusters have a crystalline structure.

11. The material particle according to claim 1, wherein said aggregate of metallic clusters has multiple stacking lattices or crystallinity.

12. The material particle according to claim 1, wherein said protective polymer layer includes small molecules, inorganic materials, or hybrid materials.

13. The material particle according to claim 1, wherein said protective polymer includes either a homo-polymer, a block polymer, a hybrid polymer, or combinations thereof.

14. The material particle according to claim 1, wherein said protective polymer is an amphiphilic polymer.

15. The material particle according to claim 1, wherein said protective polymer layer has a surface that is adaptable to include functional groups.

16. The material particle according to claim 15, wherein said functional groups include either hydrophobic groups, hydrophilic groups, or a combination thereof.

17. The material particle according to claim 16, wherein said hydrophobic groups include alkyl, aryl, or aldehyde groups.

18. The material particle according to claim 16, wherein said hydrophilic groups include hydroxyl or carboxylic groups.

19. The material particle according to claim 16, wherein said hydrophilic groups include charged sulfonate or quaternary amines groups.

20. The material particle according to claim 1, wherein said particles have a mean particle size in a range from about 1 nm to about 10 μm.

21. The material particle according to claim 20, wherein said particles have a mean particle size in a range from about 30 nm to about 900 nm.

22. The material particle according to claim 1, further having a surface with biological molecules associated by either physical or chemical mechanisms.

23. The material particle according to claim 1, wherein said particle is employed in applications involving biological detection or imaging.

24. An aqueous soluble luminescent particle comprising a luminescent metallic cluster complex encapsulated with a polymeric matrix, said luminescent metallic cluster complex having tunable structural features, said matrix being formed predominately of an organic polymer, inorganic polymer, hydrid polymer, or combinations thereof.

25. The luminescent particle according to claim 24, wherein said cluster complex has at least three metal atoms, with or without direct bonding between said metal atoms.

26. The luminescent particle according to claim 24, wherein said metal cluster hosts either an identical metal element or different metal elements.

27. The luminescent particle according to claim 24, wherein said metallic cluster complex includes Au(I)—Ag(I) polynuclei.

28. The luminescent particle according to claim 24, wherein said polymeric matrix is at least partially cross-linked with itself or with said metallic cluster complex.

29. The luminescent particle according to claim 24, wherein said polymeric matrix is surface functionalized and adapted to bind with biological molecules.

30. The luminescent particle according to claim 24, wherein said particle further incorporates at least a second kind of luminescent molecule.

31. The luminescent particle according to claim 30, wherein said second kind of luminescent molecules is at least one of the following: a different metallic cluster complex, an organic fluorescent molecule, an inorganic luminescent molecules, phosphorescent molecules, and quantum dots.

32. The luminescent particle according to claim 30, wherein said metallic cluster complex and said second kind of luminescent molecule luminesce independently of each other.

33. The luminescent particle according to claim 30, wherein said metallic cluster complex and said second kind of luminescent molecule depend upon one another to generate luminescent spectral properties.

34. The luminescent particle according to claim 30, wherein said second kind of luminescent molecule is dependent on an excitation from said metallic cluster complex to emit light.

35. The luminescent particle according to claim 30, wherein said matrix has a number of functional groups incorporated as part of said matrix and produces a functionalized exterior surface.

36. The luminescent particle according to claim 30, wherein said functional groups include at least one of the following: an amine, an alcohol, an imide, a carboxylic acid, a sulfate or sulfide, an aldehyde, an epoxy, or a combination thereof.

37. The luminescent particle according to claim 30, wherein said cluster complexes exhibit a high density of surface charges, of greater than or equal to about 5-10/nm2 (10,000/Å²).

38. The luminescent particle according to claim 37, wherein said particle has particle size that ranges from about 30 nm to about 900 nm.

39. The luminescent particle according to claim 38, wherein said particle has a mean particle size ranges from about 50 nm to about 700 nm.

40. The luminescent particle according to claim 30, wherein said particle is tagged with a biological moiety on an exterior surface.

41. The luminescent particle according to claim 40, wherein said biological moiety is attached to said exterior surface according to covalent or non-covalent bonds.

42. The luminescent particle according to claim 40, wherein said biological moiety includes antibodies, antigens, hormone molecules, nucleic acids, and proteins.

43. An optical or imaging application comprising a luminescent material particle having a metallic cluster complex doped within a polymeric matrix, said material particle having a functionalized exterior surface adapted to be either applied on or associate with at least a portion of either an inorganic or organic substrate.

44. The optical or imaging application according to claim 43, wherein said application includes an OLED display, solar photoconversion, biological assays, sensing, optical detection and imaging uses.

45. A luminescent particle comprising a luminescent core structure formed from at least one type of luminescent metal cluster complex and a shell that covers said core structure.

46. The particle according to claim 45, wherein said luminescent core structure includes one or more aggregates of said metal cluster complexes.

47. The particle according to claim 45, wherein said shell comprises one or more layers of thin films formed predominately of organic or inorganic polymers, hydrid polymers, metal film, or combinations thereof.

48. The particle according to claim 45, wherein said luminescent metal cluster complex has at least three metal nuclei, with or without direct bonding between said metal nuclei.

49. The particle according to claim 48, wherein said metal nuclei hosts either an identical metal element or different metal elements.

50. The particle according to claim 45, wherein said metallic cluster complex includes Au—Ag cluster complexes.

51. The particle according to claim 45, wherein said aggregates have at least two metal cluster complex molecules stacked together

52. The particle according to claim 45, wherein said thin films are formed by a monolayer of coating molecules

53. The particle according to claim 45, wherein said thin films are formed by multiple layers of coating molecules

54. The particle according to claim 45, wherein said polymers are co-polymers, block copolymers, non-crosslinked or at least partially cross-linked polymers and their mixtures.

55. The particle according to claim 54, wherein said polymers are attached to said metallic cluster complex core.

56. The particle according to claim 45, wherein said polymer is surface functionalized.

57. The particle according to claim 45, wherein said polymer is polymeric surfactants.

58. The particle according to claim 45, wherein said polymer is polyelectrolytes.

59. The particle according to claim 45, wherein said core structure further has at least a second type of luminescent molecule.

60. The particle according to claim 59, wherein said second kind of luminescent molecules is at least one of the following: a different metallic cluster complex, an organic fluorescent molecule, an inorganic luminescent molecule, phosphorescent molecule, and a quantum dot.

61. The particle according to claim 60, wherein said metallic cluster complex and said second kind of luminescent molecule luminesce independently of each other.

62. The particle according to claim 61, wherein said metallic cluster complex and said second kind of luminescent molecule depend upon one another to generate luminescent spectral properties through fluorescence resonant energy transfer.

63. The particle according to claim 62, wherein said second kind of luminescent molecule is dependent on an excitation from said metallic cluster complex to luminescence.

64. The particle according to claim 62, wherein said metallic cluster complex is dependent on an excitation from second kind of luminescent molecule to luminescence.

65. The particle according to claim 45, wherein said shell has a number of functional groups incorporated as part of said shell and produces a functionalized exterior surface.

66. The particle according to claim 65, wherein said functional groups include at least one of the following: an amine, an alcohol, an imide, a carboxylic acid, a sulfate or sulfide, an aldehyde, an epoxy, or a combination thereof.

67. The particle according to claim 45, wherein said particle has particle size that ranges from about 10 nm to about 15 μm.

68. The particle according to claim 65, wherein said particle has a mean particle size ranges from about 10 nm to about 5 μm.

69. The particle according to claim 45, wherein said particle is tagged with a biological moiety on an exterior surface.

70. The particle according to claim 69, wherein said biological moiety is attached to said exterior surface through covalent or non-covalent bonds.

71. The particle according to claim 69, wherein said biological moiety includes antibodies, antigens, hormone molecules, nucleic acids, and lectins.

72. The particle according to claim 45, wherein said particle is used for biological detections and imaging.

73. A luminescent metal cluster complex particle comprising more than one kind of luminescent metal cluster, or a non-metal cluster luminescent molecule, each of said metal clusters exhibiting luminescence either independent of each other, or dependent upon one another to generate luminescent spectral properties through fluorescence resonant energy transfer.

74. A method for fabricating a luminescent metal cluster particle, the method comprises: (1) dispersing a luminescent metal cluster complex in a solution suspension mixture; (2) mixing said solution suspension mixture with a polymer or co-polymer solution for a period of time to let polymer or co-polymers to coat said luminescent metal cluster complex and form a core-shell structure; (3) separating said polymer or co-polymer coated luminescent metal cluster complex particles; (4) washing the formed particles; and (5) drying the particles or suspending the particles in storage solvents.

75. A method of making a luminescent particle, said method includes the steps of: (1) uniformly dispersing more than one kind of different luminescent metal cluster complexes in a solution suspension mixture; (2) mixing the solution suspension mixture with polymers or co-polymers solution to let polymers or co-polymers adhere to and cover more than one luminescent metal cluster complexes to form a core-shell structure; (3) separating the polymer or co-polymer-coated luminescent metal cluster complex particles; (4) washing the formed particles; and (5) drying the particles or suspending the particles in storage solvents.

76. A method of making luminescent metal cluster complex particles, the method comprises: (1) dissolving a non-metallic cluster luminescent molecule in a dissolving polar solvent for form a solution I; (2) dispersing luminescent metal cluster complex in solution I to form a luminescent metal cluster complex/luminescent molecule suspension mixture II; (3) mixing said suspension mixture II with polymers or co-polymers solution to let polymers or co-polymers cover said luminescent metal cluster complex/luminescent molecule aggregate to form a core-shell structure; (4) separating said polymer or co-polymer coated luminescent metal cluster complex/luminescent molecule particles; (5) washing said formed particles; and (6) drying said particles or suspending said particles in storage solvents.

77. A biological assay application using a particle having a luminescent metallic cluster complex constituted of at least two metal cluster molecules, encapsulated within a protective polymer coating; said metallic cluster complex having a core forming an aggregate of metal atoms.

78. The biological assays according to claim 77, wherein said assay is used in detection of bacterial infection, yeast infection, viral infection, parasite infection, cancer, cardiac disease, liver disease, genetic diseases, and immunological diseases.

79. The biological assay according to claim 77, wherein said assay involves two or more different molecules, or two or more regions on a given molecule that is simultaneously detected in a sample.

80. The biological assay according to claim 77, wherein said assay involves detecting a nucleic acid in a sample.
